SEQUENCE LISTING (18 sequences)

SEQ ID NO: 1 - DNA, 44 bp, Homo sapiens
  misc_feature: human leukocyte antigen E
gggaagcttcgcgacatgggtggtggcggttctggtggtggcgg

SEQ ID NO: 2 - DNA, 44 bp, Homo sapiens
  misc_feature: human leukocyte antigen E
tagtggcggcggaggaagcggtggtggcggttccggagctcggg

SEQ ID NO: 3 - DNA, 24 bp, Homo sapiens
  misc_feature: human leukocyte antigen E
cccgagctccggaaccgccaccac

SEQ ID NO: 4 - DNA, 33 bp, Homo sapiens
  misc_feature: human leukocyte antigen E
gggaagctttgagccgagatgtctcgctccgtg

SEQ ID NO: 5 - DNA, 37 bp, Homo sapiens
  misc_feature: human leukocyte antigen E
cccagcttcagcgtacgccaaagattcaggtttactc

SEQ ID NO: 6 - DNA, 32 bp, Homo sapiens
  misc_feature: human leukocyte antigen E
atgtcgcgatcccacttaactatcttgggctg

SEQ ID NO: 7 - DNA, 31 bp, Homo sapiens
  misc_feature: human leukocyte antigen E
ggttccggatctcactccttgaagtatttcc

SEQ ID NO: 8 - DNA, 32 bp, Homo sapiens
  misc_feature: human leukocyte antigen E
gggtctagattacaagctgtgagactcagacc

SEQ ID NO: 9 - DNA, 64 bp, Homo sapiens
  misc_feature: human leukocyte antigen E
cgcttcctccgccgccactaccgccaccaccagaaccgccaccacccatgtcgcgaagcttccc

SEQ ID NO: 10 - DNA, 69 bp, Homo sapiens
  misc_feature: human leukocyte antigen E
accggatccgccaccacctaaaattaaagtccgcggagccataacagcctcgaggccagaaagagagag

SEQ ID NO: 11 - DNA, 63 bp, Homo sapiens
  misc_feature: human leukocyte antigen E
cccaagcttggcggatccggtggtggcggttctggtggtggcggctccatccagcgtacgcca

SEQ ID NO: 12 - DNA, 63 bp, Homo sapiens
  misc_feature: human leukocyte antigen E
tggcgtacgctggatggagccgccaccaccagaaccgccaccaccggatccgccaagcttggg

SEQ ID NO: 13 - DNA, 1524 bp, Homo sapiens
  misc_feature: human leukocyte antigen E-Single Chain Trimer
aagctttgagccgagatgtctcgctccgtggccttagctgtgctcgcgctactctctctttctggcctcgaggctgttatggctccgcggactttaattttaggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgtaatctaga

SEQ ID NO: 14 - DNA, 1452 bp, Homo sapiens
  misc_feature: human leukocyte antigen E-Single Chain Dimer
aagctttgagccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgtaatctaga

SEQ ID NO: 15 - protein, 500 aa, Homo sapiens
  MISC_FEATURE: human leukocyte antigen E Single chain Trimer
MSASVALAVLALLSLSGLGAVMAPATLILGGGGSGGGGSGGGGSIGATPLIGVTSAHPAGAGLSAPLACTVSGPHPSAIGVALLLAGGAIGLVGHSALSPSLATSPTLLTTTGPTPTGLAGTACAVAHVTLSGPLIVLTAAAMGGGGSGGGGSGGGGSGGGGSGSHSLLTPHTSVSAPGAGGPAPISVGTVAATGPVAPAAAAASPAMVPAAPTMGGGGSGTTAAGTASAAATAGIPAVALATLAGTTAGSGAGSHTLGTMHGCGLGPAAAPLAGTGGPATAGLATLTLAGALASTTAVATAAGISGGLSAAASGAGHGAATLGATCVGTLHLTLGLGLGTLLHLGPPLTHVTHHPISAHGATLACTALGPTPAGITLTTGGAGGGHTGATGLVGTAPAGAGTPGLTAAVVVPSGGGGATTCHVGHGGLPGPVTLATLPASGPTIPIVGIIAGLVLLGSVVSGAVVAAVITALLSSGGLGGSTTLAGTSASAGGSGSHSL

SEQ ID NO: 16 - protein, 476 aa, Homo sapiens
  MISC_FEATURE: human leukocyte antigen E-Single chain dimer
MSASVALAVLALLSLSGLGAIGATPLIGVTSAHPAGAGLSAPLACTVSGPHPSAIGVALLLAGGAIGLVGHSALSPSLATSPTLLTTTGPTPTGLAGTACAVAHVTLSGPLIVLTAAAMGGGGSGGGGSGGGGSGGGGSGSHSLLTPHTSVSAPGAGGPAPISVGTVAATGPVAPAAAAASPAMVPAAPTMGGGGSGTTAAGTASAAATAGIPAVALATLAGTTAGSGAGSHTLGTMHGCGLGPAAAPLAGTGGPATAGLATLTLAGALASTTAVATAAGISGGLSAAASGAGHGAATLGATCVGTLHLTLGLGLGTLLHLGPPLTHVTHHPISAHGATLACTALGPTPAGITLTTGGAGGGHTGATGLVGTAPAGAGTPGLTAAVVVPSGGGGATTCHVGHGGLPGPVTLATLPASGPTIPIVGIIAGLVLLGSVVSGAVVAAVITALLSSGGLGGSTTLAGTSASAGGSGSHSL

SEQ ID NO: 17 - protein, 9 aa, Homo sapiens
  MISC_FEATURE: human leukocyte antigen E
VMAPRTLIL

SEQ ID NO: 18 - protein, 9 aa, Homo sapiens
QMRPVSRVL

(19) United States
(12) Patent Application Publication
(10) Pub. No.: US 2005/0196404 A1
(43) Pub. Date: Sep. 8, 2005

(54) Genes encoding single chain human leukocyte antigen E (HLA-E) proteins to prevent natural killer cell-mediated cytotoxicity and cytotoxic T Lymphocyte (CTL)-mediated cytotoxicity

(76) Inventor: Mark D. Crew, Little Rock, AR (US)

Correspondence Address: BUTLER, SNOW, O'MARA, STEVENS & CANNADA PLLC, 6075 POPLAR AVENUE, SUITE 500, MEMPHIS, TN, 38119, US

(21) Appl. No.: 11/109,435

(22) Filed: Apr. 19, 2005

Related U.S. Application Data
(63) Continuation-in-part of application No. 10/430,984, filed on May 6, 2003.

(51) Int. Cl. A61K 39/395; A61K 39/00

(52) U.S. Cl. 424/185.1

(57) ABSTRACT

The present invention describes a gene encoding a single polypeptide encoding three components of HLA-E cell surface expression. When the gene is introducted into porcine cells it fold properly and confers protection against human NK cell-mediated killing. Additionally, this invention provides a method to inhibit CTL-mediated killing of cells.

### CROSS-REFERENCE TO RELATED APPLICATIONS

This application claims priority under 35 U.S.C. §120 as a CONTINUATION IN PART APPLICATION of a co-pending application entitled “Genes Encoding Single Chain Human Leukocyte Antigen E (HLA-E) Proteins to Prevent Natural Killer Cell-Mediated Cytotoxicity” which was filed on May 6, 2003, and was assigned U.S. application Ser. No. 10/430,984 (the “'984 application”), the entire disclosure of which is incorporated herein by reference for all that it teaches.

### STATEMENT REGARDING FEDERALLY SPONSORED RESEARCH OR DEVELOPMENT

The invention was made with Government support under the terms of A149885 awarded by NIH/NIAID and the Office of Research and Development, Department of Veterans Affairs. The Government has certain rights in the invention.

### BACKGROUND OF THE INVENTION

1. Field of the Invention

The present invention relates to genetic technology to eliminate human natural killer (NK) cell and cytotoxic T Lymphocyte (CTL)-mediated rejection of xenografts.

2. Brief Description of the Related Art

Pig-to-human xenotransplantation is an attractive means to alleviate the critical shortage of human organs. Human natural killer (NK) cells, although not generally considered significant in allotransplants, may play an important role in the rejection of porcine xenografts.

Several lines of evidence suggest that NK cells participate in pig-to-primate xenograft rejection. First, there are numerous reports describing the killing of cultured pig cells by human NK cells (Seebach et al. 1996; Chan & Auchincloss, 1996; Donnelly et al. 1997; Matter-Reissmann et al. 2002, Xenogeneic human NK cytotixicity against porcine endothelial cells is perforin/granzyme B dependent and not inhibited by Bcl-2 overexpression. Xenotransplantation 9:325-337; Horvath-Arcidiacono & Bloom; 2003). Second, recipient NK cell infiltration has been observed in pig kidney grafts undergoing acute vascular rejection (AVR) in a pig-to-cynomolgus monkey model and more infiltrating NK cells were observed in grafts undergoing AVR than in grafts without AVR (Quan et al. 2000, Identification, detection, and in vitro characterization of cynomolgus monkey natural killer cells in delayed xenograft rejection of hDAF transgenic porcine renal xenografts. Transplant. Proc. 32:936). Finally, human NK infiltration of pig kidneys is also seen in an ex vivo perfusion model using human blood (Khalfoun et al., 2000, Development of an ex vivo model of pig kidney perfused with human lymphocytes. Analysis of xenogeneic cellular reactions. Surgery 128:447457).

NK cells are a key component of the innate immune system and influence adaptive immune responses via cytokine secretion. The activity of NK cells is thought to be controlled by the balance of inhibitory and activating signals delivered via NK cell cell-surface receptors (Lopez-Botet & Bellon 1999, Natural killer cell activation and inhibition by receptors for MHC class I; Curr Opin Immunol. 11:301-307). Conceivably then, eliminating ligands for NK cell activation receptors on pig cells or increasing the level of ligands for inhibitory cell receptors could abrogate human NK cell-mediated destruction of porcine xenografts. The latter strategy has received the most attention probably due to the scant understanding of porcine NK cell activating ligands.

There are two classes of NK cell inhibitory receptors: the immunoglobulin-like KIR and LIR receptors and the C-type lectin-like receptors (CD94/NKG2 heterodimers). In humans, the ligands for the KIR receptor family members are the classical class I antigens, HLA-A, -B, and -C and the ligand for some LIRs (LIR-1 and -2) is the nonclassical class I antigen HLA-G. The major ligand for CD94/NKG2 receptors is the nonclassical class I antigen HLA-E. Ligands for several types of human NK cell inhibitory receptors have been expressed in pig cells and tested for their ability to modulate NK cell activity.

When ligands for KIRs, specifically HLA-A2, -B27, and -Cw3, were expressed in immortalized porcine endothelial cells, only HLA-Cw3 conferred protection against lysis by human NK cells but only if the NK cells expressed CD158b; protection against lysis by NK cells expressing CD158a was not observed (Seebach et al. 1997, HLA-Cw3 expression on porcine endothelial cells protects against xenogeneic cytotoxicity mediated by a subset of human NK cells; J. Immunol. 159:3655-3661). Utilization of a classical class I antigen such as HLA-Cw3 is problematic insofar as the induction of alloreactive T cells may occur. Mutation of the CD8 binding site of HLA-Cw3 (D227K) ameliorated its potential alloreactivity but consistent with the results of Seebach et al. (1997; J. Immunol. 159:3655-3661), complete protection to lysis by CD158b+ NK cells but only partial protection against lysis by polyclonal NK cell preparations was observed (Sharland et al., 2002, Genetically modified HLA class I molecules able to inhibit human NK cells without provoking alloreactive CD8+CTLs. J. Immunol. 168:3266-3274).

HLA-G has been explored as a potential inhibitor of human NK cell lysis of pig cells with mixed results. An early report describes dramatic decreases in the ability of human NK cells to lyse porcine aortic endothelial cells transfected with HLA-G (Sasaki et al. 1999, HLA-G expression protects porcine endothelial cells against natural killer cell-mediated xenogeneic cytotoxicity; Transplantation 67:31-37). However, results from other studies suggest that HLA-G either only partially protects against human NK cell-mediated cytotoxicity (Forte et al., 2001, HLA-G inhibits rolling adhesion of activated human NK cells on porcine endothelial cells. J. Immunol. 167:6002-6008; Matsunami et al. 2002, Modulation of the leader peptide sequence of the HLA-E gene up-regulates its expression and down-regulates natural killer cell-mediated swine endothelial cell lysis; Transplantation 73:1582-1589) or fails completely (Dorling et al., 2000, HLA-G inhibits the transendothelial migration of human NK cells; Eur J Immunol. 30:586-593) although human NK cell/porcine endothelial cell interaction may be appreciably altered (Dorling et al., 2000, Eur J. Immunol. 30:586-593; Forte et al., 2001, J. Immunol. 167:6002-6008).

Among NK cell inhibitory receptors, CD94/NKG2A appears to be widely expressed among NK cells. Thus, the ligand for CD94/NKG2A, HLA-E, when expressed on porcine cells might be the most potent inhibitor of human NK cell lysis. The cell-surface expression of HLA-E on pig cells is somewhat controversial. (Sasaki et al. 1999, HLA-E and HLA-G expression on porcine endothelial cells inhibit xenoreactive human NK cells through CD94/NKG2-dependent and -independent pathways; J. Immunol. 163:6301-6305) report that transfection of the HLA-E gene together with the human β2-microglobulin (β2m) gene resulted in readily detectable cell-surface expression of HLA-E and conferred a 34-84% reduction in NK cell-mediated killing of porcine endothelial cells. (Matsunami et al. 2002; Transplantation 73:1582-1589), on the other hand, detected HLA-E cell-surface expression on transfected porcine endothelial cells only when a canonical HLA-E binding peptide was endogenously added. The canonical HLA-E binding peptide is found in the leader peptide of HLA-A, -B, -C, and -G proteins (Braud et al., 1998, TAP- and tapasin-dependent HLA-E surface expression correlates with the binding of an MHC class 1 leader peptide; Curr Biol. 8:1-10) and HLA-E expression was also detected when the HLA-E gene was co-transfected with the HLA-G gene or when the leader peptide-encoding sequence of HLA-E was replaced with the corresponding sequences of HLA-A2 or HLA-G. The discrepancy regarding cell-surface expression of HLA-E might be due to the difference in the strains of pigs from which the endothelial cells were derived. That is, an HLA-E binding peptide may be expressed in one strain but not another. Pig strains expressing an HLA-E binding peptide might be quite rare as cell-surface of expression of transfected HLA-E was not observed in three additional, independently-derived porcine cell lines (M.D.C, unpublished observations).

The binding of HLA-E to CD94/NKG2A, and subsequent negative signaling is highly dependent on the nature of the peptide bound to HLA-E and the HLA class I signal sequence-derived peptides are optimal in this regards. Although not rigorously examined, human β2m may also be required for maximal cell-surface expression in pig cells. Generating pigs transgenic for three genes (HLA-E heavy chain, human β2m, and some gene encoding an HLA-E binding peptide) in order to ensure HLA-E cell-surface expression is technically difficult and would be tedious. While the leader peptide of HLA-E could be replaced by one containing a canonical HLA-E binding peptide, the level of peptide produced may not be sufficient to keep HLA-E bound solely with that peptide.

Yu et al. described a single chain trimer of a mouse classical class I protein (H-2 K^{b}) in which the peptide antigen (“OVA”) bound to the heavy chain is covalently attached by a fifteen amino acid peptide linker to mouse β2m which is itself attached to the Kb heavy chain by a twenty amino acid peptide linker (Yu et al., 2002, Cutting Edge: single-chain trimers of MHC class I molecules form stable structures that potently stimulate antigen-specific T cells and B cells; J. Immunol. 168:3145-3149). The OVA peptide was shown to be extraordinarily tightly bound to the Kb heavy chain. Moreover, the single chain OVA-β2m-Kb trimer was able to induce OVA-specific, Kb-restricted T cell responses.

There exits a need in the art to circumvent having to separately express human β2m and an HLA-E binding peptide in order to achieve HLA-E cell-surface expression. The invention herein describes the construction of a single chain trimer (SCT) of HLA-E. Furthermore, the invention also describes the expression and functional analysis of the HLA-E SCT in pig cells.

References mentioned in this background section are not admitted to be prior art with respect to the present invention.

### BRIEF SUMMARY OF THE INVENTION

The present invention relates in part to methods to use a ligand that binds to natural killer (NK) cell killer inhibitory receptors. The ligand is human leukocyte antigen E (HLA-E). HLA-E on the cell surface is a trimer of three polypeptides: the HLA-E heavy chain (encoded by HLA-E gene), beta-2 microglobulin (β2m), and a nine amino acid peptide usually derived from the leader sequence (single peptide) of other HLA class I proteins (HLA-A, -B, -C, or -G). All three components of the HLA-E trimer are required for HLA-E cell-surface expression. The nucleotide sequence of the HLA-E single chain trimer gene is set out in SEQ. ID. NO. 13. The gene shown in SEQ. ID. NO. 13 encodes a single polypeptide (SEQ. ID. NO. 15) made of all three components of HLA-E cell surface expression. When introduced into pig cells the polypeptide shown in SEQ. ID. NO. 15 folds properly and confers protection against human NK cell mediated killing.

This invention provides methods of promoting tolerance and inhibiting NK cell mediated attack in a human recipient to a swine graft. These methods include introducing into the recipient a swine biological material, such as a swine hematopoietic stem cell which has been transformed with a transgene encoding a HLA-E single chain trimer polypeptide that inhibits recipient NK cell mediated attack. More specifically, this invention relates to a method to prevent Natural Killer cell-mediated rejection of a xenograft. This method includes the steps of: (a) providing an isolated and purified HLA-E polypeptide comprising the amino acid sequence of SEQ. ID. LISTING NO. 15; (b) administering to cells or whole animals said polypeptide; and (c) producing an inhibitory response to said natural killer cell-mediated rejection of a xenograft. More specifically, this invention provides a method of inducing at least partial immunological tolerance in a recipient human to a graft obtained from a donor swine. The method includes the steps of: (a) introducing into the recipient human swine biological material including a transgene encoding an isolated and purified HLA-E polypeptide comprising the amino acid sequence of SEQ ID LISTING NO. 15; (b) implanting the swine graft into the recipient human, wherein at least one of the cells of the swine graft express HLA-E polypeptide; and (c) wherein the introduction of swine biological material into the recipient human results in at least partial immunological tolerance to the swine graft.

The receptor for HLA-A is CD94/NKG2A. Once thought to exist only on NK cells, it is now known to be expressed on CD8+ T cells (cytotoxic T lymphocytes, CTLs) following antigenic stimulation with viruses or bacteria. We now show that CD94/NKG2A is induced on human CTLs following xenogenic stimulation (i.e. after co-incubation with pig aortic endothelial cells, PAECs) and moreover that PAECs expressing the HLA-E single chain trimer are significantly less susceptible to xenoreactive CD8+ CD94/NKG2A+ T cells. Thus, the invention further relates to a method to inhibit CTL-mediated killing of cells including the steps of: (a) providing an isolated and purified HLA-E polypeptide made of the amino acid sequence of SEQ ID LISTING NO. 15; (b) administering to cells or whole animals the protein; and (c) producing an inhibitor response to said CTL-mediated killing of cells.

More specifically, this invention relates to a method of inducing at least partial CTL mediated immunologic tolerance in a recipient human to human donor cell, the method comprising: (a) transfecting a human donor cell with isolated and purified HLA-E single chain trimer polypeptide made of the amino acid sequence of SEQ ID LISTING NO. 15 to provide transfected cells; and (b) introducing into the recipient human the transfected cells; wherein the introducing of said transfected cells into the recipient human results in at least partial CTL mediated immunologic tolerance to the transfected cells.

In another embodiment the invention relates to the use of the polynucleotide, the vector, the antibody and/or anti-idiotype antibody of the present invention for the preparation of a pharmaceutical composition for preventing and/or treating a natural killer or T cell-mediated cytotoxicity.

Additionally, this invention also relates to the use of the antagonist, the polynucleotide, the vector, the antibody and/or the anti-idiotype antibody of the invention for the investigation of HLA-E receptor type-specific functions.

The present invention also relates to a pharmaceutical composition made of the polynucleotide, the vector, the antibody and/or the anti-idiotypic antibody of the present invention and optionally a pharmaceutically acceptable carrier and/or diluent.

Examples of suitable pharmaceutical carriers are well known in the art and include phosphate buffered saline solutions, water, emulsions, such as oil/water emulsions, various type of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. The dosage regimen will be determined by the attending physician and clinical factors. As it well known in the medical arts, dosages for any one patient depends upon many factors, including the patient's size, body surface area, age, the particular compound to be administered, sex, time and route of administration, general health, and other drugs being administered concurrently. The compositions of the invention may be administered locally or systemically. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chroride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition.

The present invention further relates to a kit comprising the polynucleotide, the vector, the antibody and/or the anti-idiotypic antibody of the present invention.

The documents cited herein are herewith incorporated by reference.

### BRIEF DESCRIPTION OF THE DRAWINGS

These and other features, objects and advantages of the present invention will become better understood from a consideration of the following detailed description and accompanying drawings.

FIG. 1 shows a schematic of HLA-E SCT and single chain dimer (SCD) structures. Human β2m-encoding sequences, including those encoding the signal peptide (s.p.), are shown as double hatched. HLA-E sequences are shown as hatched and the peptide antigen-encoding region of HLA-E SCT is white. The positions and composition of connecting peptides are given above each construct and the peptide sequences of two HLA-E SCTs examined are shown below the HLA-E SCT structure.

FIG. 2A-2H show transient transfection analysis of HLA-E expression in porcine epithelial cells. The indicated cDNAs (all under control of the CMV immediate-early promoter) were co-transfected with pEGFP-C1 (encoding enhanced green fluorescence protein). Forty eight hours post-transfection, cells were harvested, stained with the HLA-E-specific mAb 3D12 (panels A-D, G, and H) or the HLA-B27 specific mAb HLA.ABC.m3 (panels E and F) and analyzed by flow cytometry, with gating on EGFP positive cells. The HLA-E or HLA-B27 straining of EGFP-positive cells is shown (dark curves); isotype control mAb staining is designated by dashed curves.

FIG. 3A-3F show flow cytometric analyses of sorted HLA-E SCT LLC-PK1 cell stable transfectants. Untransfected LLC-PK1 cells (light curves) or E-Cw*03 SCT transfected LLC-PK1 cells which were sorted based on 3D12 staining (dark curves) were analyzed by flow cytometry using the mAbs listed above each histogram.

FIG. 4 shows NK cell-mediated lysis of untransfected and Cw*03 SCT transfected LLC-PK1 cells. Shown are the percent specific lysis of untransfected LLC-PK1 cells (filled circles) and E-Cw*03 SCT transfected LLC-PK1 cells (open circles) at 4 hours at various effector:target ratios (upper graphs) and at an effector:target ratio of 10:1 for various times (lower graphs) by NK cell lines NK-92 and NKL (as indicated above the graphs). Representative results from over six experiments are shown.

FIG. 5 shows IFN-γ secretion by co-cultured NKL cells. The concentration of IFN-γ in supernatants of LLC-PK1 cells alone (“LLCPK1”), LLC-PK1 cells co-cultured with NKL cells (“LLCPK1+NKL”), HLA-E SCT transfected LLC-PK1 cells co-cultured with NKL cells (“E-SCT+NKL”), and NKL cells cultured alone (“NKL”) is shown. Representative results from four separate experiments are shown with error bars designating the range of triplicate values in one experiment.

FIG. 6A-B show the peptide antigen-dependence of HLA-E SCT in NK cell recognition/lysis. A. FACS analysis of E-Cw*03 SCT and E-hsp60 SCT LLK-PK1 cell stable transfectants. FACS analysis of LLC-PK1 cells stably transfected with E-Cw*03 SCT-(dark curve), E-shp60 SCT-(light curve), and vector-(dashed curve) transfected LLC-PK1 cells using the conformation dependent, HLA-E-specific mAb MEM-E/6 as primary antibody is shown. B. Susceptibility of HLA-E SCT transfectants to NK cell mediated lysis. The percent specific lysis (\tilde{y}axis) observed in 4 h cytotoxicity assays using NKL cells as effectors and as targets, LLC-PK1 cells transfected with vector (closed circles, ∘), E-Cw*03 SCT (open circles, ∘), or E-hsp60 SCT (open squares, □) is shown. Typical results from more than five separate experiments are shown.

FIG. 7 is a schematic depiction of HLA-trimeric complex and HLA-E single chain trimer.

FIG. 8 is an HLA-E single chain trimer gene.

FIG. 9 is a FACs analysis of HLA-E single chain trimer expression on LLC-PK1 cell surface. Dashed curves indicate staining of untransfected LLC-PK1 cells. Solid curves indicate staining of HLA-E single chain trimer gene-transfected LLC-PK1 cells.

FIG. 10 is a FACs analysis of HLA-E single chain trimer expression on LLC-PK1 cell surface. Dashed curves indicate staining of untransfected LLC-PK1 cells. Solid curves indicate staining of HLA-E single chain trimer gene-transfected LLC-PK1 cells.

FIG. 11 CD94/NKG2A induction on CB8+ T cells following xenoreactive stimulation and inhibition of xenoreactive CB8+ T cell-mediated lysis by HLA-E SCT-expressing AOC cells. Shown is NKG2A cell-surface expression on CD8+ cells dashed curves are CD8+ cells co-cultured with AOC cells; solid curves are CD8+ T cells dashed cultured by themselves). Xenoreactive CD8+ T cells were used as effector's in cytotoxicity assays using untransfected AOC cells (open circles) of AOC cells stably transfected with HLA-E SCT (filled squares).

### DETAILED DESCRIPTION OF THE INVENTION

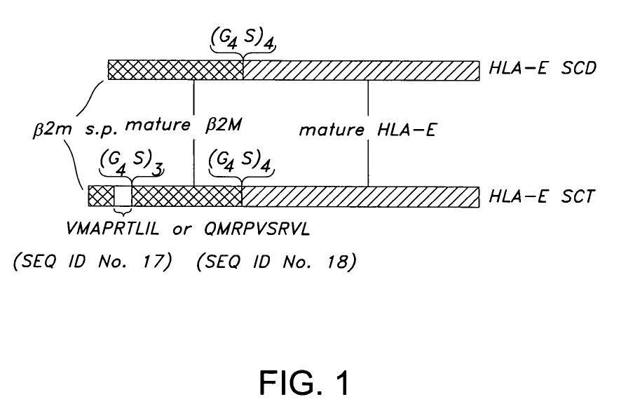

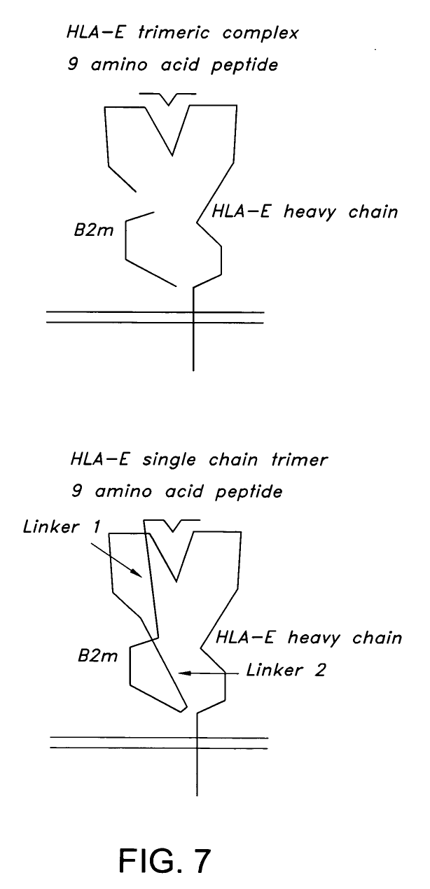

With reference to FIGS. 1-11, the preferred embodiment of the present invention may be described. The present invention is directed to satisfying the need for a single chain trimer gene which folds properly and confers protection against human NK-cell mediated killing.

The HLA-E single chain trimer (SCT) gene and, as a control, and an HLA-E single chain dimer (SCD) gene (i.e. lacking peptide antigen-encoding sequences) were constructed. A schematic depiction of HLA-E SCD and SCT genes is shown in FIG. 1. E-Cw*03 SCT is made of the signal peptide-encoding portion of the human B2m gene followed by a sequence encoding a canonical HLA-E binding peptide antigen, VMAPRTLIL (SEQ ID NO. 17) which is identical to that found in the signal peptides of HLA-Cw*03 (and 35 other HLA-C alleles of the 63 for which the signal peptide sequence is known) and HCMV UL40 (33,34) (Tomasec P., V. M. Braud, C. Rickards, M. B. Powell, B. P. McSharry, S. Gadola, V. Crumdolo, L. K. Forysiewicz, A. J. McMichael, G. W. Wilkinson. 2000. Surface expression of HLA-E, an inhibitor of natural killer cells, enhanced by human cytomegalovirus gpUL40. Science 287:1031.) (Ulbretch M., S. Martinozzi, M. Grzeschik, H. Hengel, J. W. Ellwart, M. Pla, E. H. Weiss. 2000. Cutting edge: the human cytomegalovirus UL40 gene product contains a ligand for HLA-E and prevents NK cell mediated lysis. J. Immunol. 164:50(19). The peptide antigen encoding-sequence is followed by a 45 bp sequence encoding “connecting peptide 1” which when translated will yield the 15 amino acid sequence (G₄S)₃. Immediately 3′ to connecting peptide I-encoding DNA is the sequence for mature (lacking signal peptide) human β2m cDNA which is linked to the sequence of mature HLA-E heavy chain by a 60 bp sequence encoding “connecting peptide 2” which when translated will yield the 20 amino acid sequence (G₄S)₄. The structure of HLA-E SCD is similar to that of E-Cw*03 SCT except HLA-E SCD lacks sequences encoding peptide antigen and connecting peptide 1 and the signal peptide of human β2m is in its natural location (FIG. 1). [The nucleotide sequences of E-Cw*03 SCT and HLA-E SCD genes have been deposited in GenBank (accession numbers AY289236 and AY289237, respectively)].

Cell-Surface Expression of HLA-E SCD and SCT Proteins.

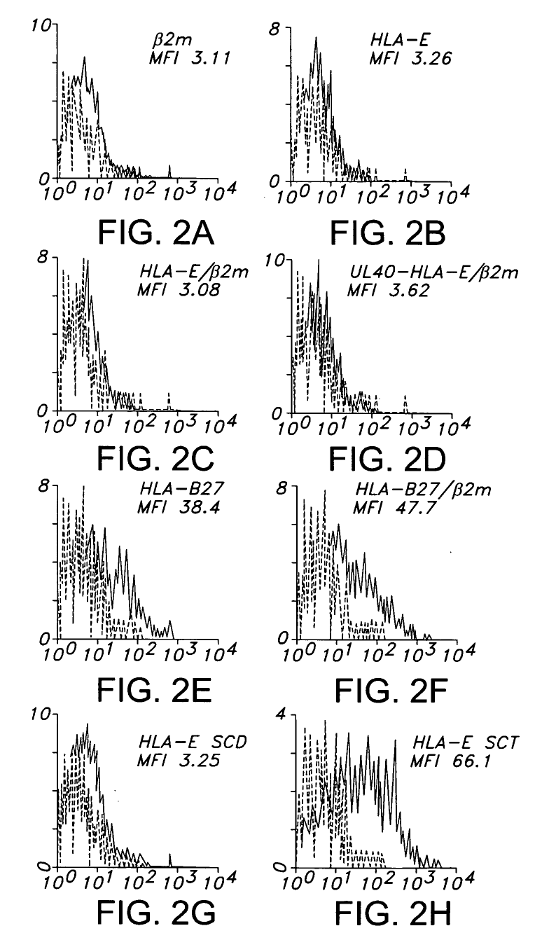

Initial assessment of E-Cw*03 SCT cell-surface expression utilized transiently transfected LLC-PKI cells. Significant 3D12 staining, indicative of HLA-E ce11-surface expression, was observed in E-Cw*03 SCT transfectants (FIG. 2) while HLA-E SCD transfectants (FIG. 2) exhibited 3D12 staining comparable to that seen in LLC-PK1 cells transfected with just human β2m (FIG. 2) or just vector alone. These findings suggest that the covalently attached peptide antigen of E-Cw*03 SCT markedly enhances cell-surface expression.

LLC-PKI cells were stably transfected with E-Cw*03 SCT and HLA-E SCD and examined for HLA-E expression, again using the HLA-E specific mAb 3012. Substantial HLA-E cell-surface expression was observed in E-Cw*03 SCT transfectants while no 3D12 staining was observed in LLC-PKI cells stably transfected with vector alone. LLC-PK I cells stably transfected with HLA-E SCD, unlike those transiently transfected with this construct, showed detectable levels of HLA-E cell-surface expression. However, the mean fluorescent intensity (MFI) of 3DI2 staining of HLA-ESCD transfected cells was noticeably reduced compared to E-Cw*03 SCT stable transfectants (MFI of 62 versus 375, respectively. Such results are consistent with the idea that the covalently attached peptide antigen in E-Cw*03 SCT significantly increases the stability of the HLA-E SCT. However, for reasons not entirely clear, addition of exogenous VMAPRTLIL peptide at concentrations as high as 300 uM did not increase HLA-E SCD cell-surface expression even when peptide loading was performed on cells grown at 26° C. (data not shown).

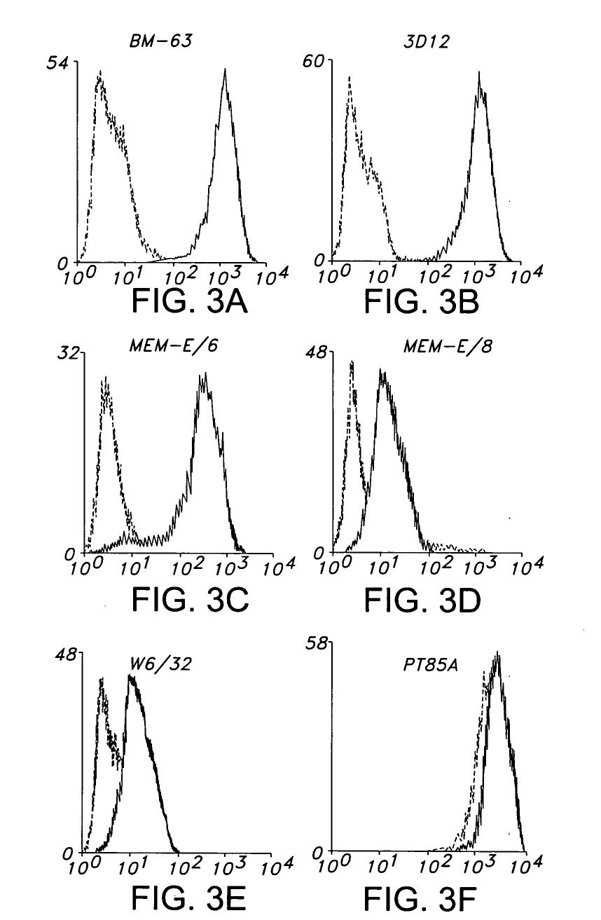

A homogenous population of E-Cw*03 SCT positive LLC-PKI cells was obtained by fluorescent activated cell sorting using mAb 3D12. These were analyzed by flow cytometry with an expanded panel of specific mAbs (FIG. 3). The cell sorting was effective and efficient in that 100% of the cells were stained with the HLA-E specific mAb 3DI2 (FIG. 3B). The cells were also all positive for BM-63 (FIG. 3A), a mAb specific for human B2m. mAb BM-63 is not only human-specific but its binding is also conformational dependent; the high MFI observed thus indicated that at least the B2m domain of HLA-E SCT is folded correctly. Two additional HLA E-specific mAbs, MEM-E/6 and MEM-E/8 (Menier C., B. Saez, V. Horejsi, S. Martinozzi, I. Krawice-Radanne, S. Bruel, C. Le Danff, M. Reboul, I. Hilgert, M. Rabreau, M. L. Larrad, M. Pla, E. D. Carosella, N. Rouas-Freiss. 2003. Characterization of monoclonal antibodies recognizing HLA-G or HLA-E: new tools to analyze the expression of nonclassical HLA class I molecules. Hum. Immunol. 64:315) were also examined. These mAbs are also conformation-dependent and both stained LLC-PKI cells expressing E-Cw*03 SCT (FIGS. 3C and 3D) although MEM-E/8 staining was appreciably less than MEM-E/6 staining. A pan-HLA class I-specific mAb, W6/32, which recognizes HLA-E, was also tested (FIG. 3E). E-Cw*03 SCT transfected LLC-PKI cells were uniformly positive for W6/32 although the fluorescent intensity was quite weak. The weak staining by W6/32 can be attributed to the fact that the epitope of W6/32 includes the amino terminus of human B2m (Shields M. J., R. K. Ribaudo. 1998. Mapping of the monoclonal antibody W6/32: sensitivity to the amino terminus of beta2-microglobulin. Tissue Antigens 51:567) which is not present in E-Cw*03 SCT. PT85A is a comformation dependent mAb purportedly specific to porcine MHC class I antigens (Davis W. C., S. Marusic, H. A. Lewin, G. A. Splitter, L. E. Perryman, T. C. McGuire, J. R. Gorham. 1987. The development and analysis of species specific and cross reactive monoclonal antibodies to leukocyte differentiation antigens and antigens of the major histocompatibility complex for use in the study of the immune system in cattle and other species. Vet. Immunol. Immunopathol. 15:337) but also binds at least some HLA class I antigens. PT85A stained brightly untransfected LLC-PKI cells; staining of HLA-E SCT transfected cells was slightly, but reproducibly, higher (FIG. 3F). Taken together, the flow cytometric analyses of E-Cw*03 SCT-expressing LLC-PKI cells indicate that the vast majority of E-Cw*03 SCT expressed on the cell-surface is serologically undistinguishable from correctly folded, native HLA-E.

Cell-Surface Stability of E-Cw*03 SCT.

An OVA-B2m-kb SCT exhibited remarkable cell-surface stability with a half-life of greater than 16 hours (Yu Y. Y., N. Netuschil, L. Lybarger, J. M. Connolly, T. H. Hansen. 2002. Cutting edge: single-chain trimers of MHC class I molecules form stable structures that potently stimulate antigen-specific T cells and B cells. J. Immunol. 168:3145). To assess the cell-surface half-life of E-Cw*03, LLC-PKI cells stably transfected with E-Cw*03 SCT-encoding plasmid were analyzed by FACS after treatment with Brefeldin A for various times. The HLA-E-specific mAb 3D12 was used to monitor E-Cw*03 SCT surface expression and, for comparison, mAb PT85A was used to follow cell-surface levels of endogenous pig MBC (SLA) class I cell-surface expression. The cell-surface half-life of SLA class I proteins has not been reported but if SLA class I cell-surface stability on LLC-PKI cells reflects SLA class I in general, they are relatively long-lived with a half-life of about 36 hours (data not shown). In contrast, the half-life of cell-surface E-Cw*03 SCT was only about 4 hours, far shorter than H-2 class I SCTs previously described (Yu Y. Y., N. Netuschil, L. Lybarger, J. M. Connolly, T. H. Hansen. 2002. Cutting edge: single-chain trimers of MHC class I molecules form stable structures that potently stimulate antigen-specific T cells and B cells. J. Immunol. 168:3145).

Susceptibility of Pig (LLC-PK1) Cells Expressing HLA-E SCT to Lysis by Human NK Cells.

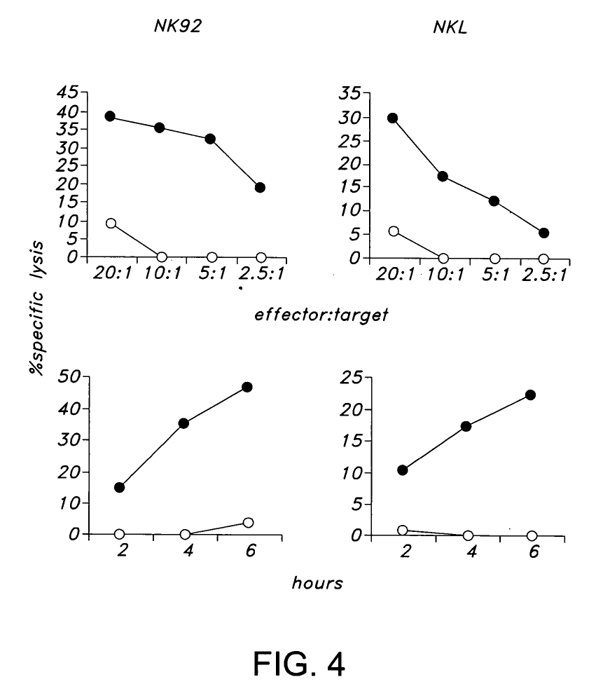

Flow cytometric analyses suggested that E-Cw*03 SCT is expressed at the cell-surface with a correct conformation (FIGS. 1H and 3A-3F). The functionality of E-Cw*03 SCT was directly assessed by testing its ability to confer protection against human NK cell-mediated lysis. Two NK cell lines, NK-92 and NKL (Robertson M. J., K. J. Cochran, C. Cameron, J. M. Le, R. Tantravahi, J. Ritz. 1996. Characterization of a cell line, NKL, derived from an aggressive human natural killer cell leukemia. Exp. Hematol. 24:406) (Gong, J. H., G. Maki, H. G. Klingemann. 1994. Characterization of a human cell line (NK-92) with phenotypical and functional characteristics of activated natural killer cells. Leukemia 8:652), were used as effectors in standard ⁵¹Cr-release assays to quantify cytotoxicity. As targets, untransfected LLC-PK I cells or LLC-PKI cells transfected with E-Cw*03 SCT were used. The results, shown in FIG. 4, clearly demonstrate that E-Cw*03 SCT protects LLC-PKI cells from killing by human NK cells. Untransfected LLC-PKI cells were specifically lysed by NK-92 cells at effector:target ratios ranging from 2.5:1 to 20:1 in a time-dependent manner (FIG. 4). In contrast, LLC-PKI cells expressing E-Cw*03 SCT were almost completely protected with only minimal lysis observed at 6 hours or at an effector:target ratio of 20:1 (FIG. 4). NKL cells lysed untransfected LLC-PKI cells to a slightly lesser degree than did NK-92 cells but the results with regards to E-Cw*03 SCT were identical—the susceptibility to lysis was virtually abolished by expression of E-Cw*03 SCT (FIG. 4). Thus, E-Cw*03 SCT, in which all three components of a normal HLA-E protein complex (heavy chain, β2m, and peptide) are in one polypeptide chain, is immunologically functional in terms of its ability to modulate NK cell cytotoxicity.

IFN-γ Secretion of Human NK Cells in Response to LLC-PKI Cells Expressing E-Cw*03 SCT.

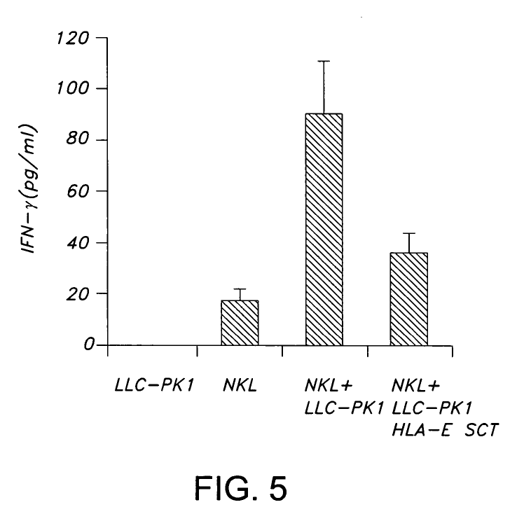

NK cells participate in the innate immune response not only by their cytolytic activity but also by their secretion of cytokines, IFN-γ in particular, which can attract and activate other cells of the innate and adaptive immune systems (Biron, C. A., K. B. Nguyen, G. Pien, C. Cousens, T. P. Salazar-Mather. 1999. Natural killer cells in antiviral defense: function and regulation by innate cytokines. Annu. Rev. Immunol., 17:189) (Boehm, U., T. Klamp, M. Groot, J. C. Howard, 1997. Cellular responses to interferon gamma. Annu. Rev. Immunol. 15:749). The ability of E-Cw*03 SCT to alter NK cell IFN-γ secretion was therefore examined by ELISA. NKL cells were cultured alone or co-cultured with untransfected LLC-PKI cells or LLC-PKI cells expressing E-Cw*03 SCT. LLC-PKI cells by themselves served as a negative control. After 48 hours of co-culture, supernatants were collected and assayed. NKL cells co-cultured with untransfected LLC-PKI cells secreted about four-fold more IFN-γ than NKL cells cultured alone (FIG. 5). NKL cell IFN-γ secretion when co cultured with LLC-PKI cells expressing E-Cw*03 SCT was nearly equivalent to that observed with NKL cells alone (FIG. 5). Thus, E-Cw*03 SCT appears to also prohibit human NK cell cytokine secretion incurred by contact with pig cells.

Peptide Dependency of HLA-E SCT in Conferring Protection Against NK Cell Cytotoxicity.

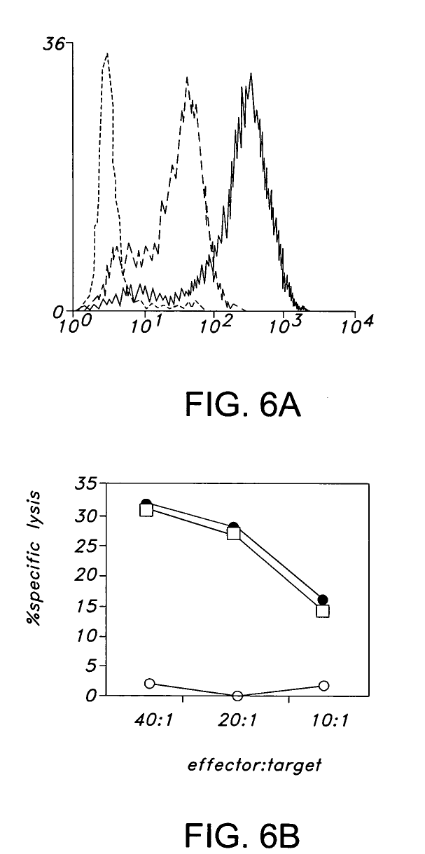

Michaelsson et al. (2002) reported that an hsp60-derived peptide (QMRPVSRVL) (SEQ Id. No. 18) is able to bind HLA-E and stabilize HLA-E cell-surface expression (Michaelsson J., C. Teixeira de Matos, A. Achour, L. L. Lanier, K. Karre, K. Soderstrom. 2002. A signal peptide drived from hsp60 binds HLA-E and interferes with CD94/NKG2A recognition. J. Exp. Med. 196:1403). However, HLA-E complexed with QMRPVSRVL (SEQ. ID. NO. 18) was unable to bind CD94/NKG2A and as a consequence, failed to protect against NK cell-mediated cytotoxicity (Michaelsson J., C. Teixeira de Matos, A. Achour, L. L. Lanier, K. Karre, K. Soderstron. 2002. A signal peptide derived from hsp60 binds HLA-E and intereferes with CD94/NKG2A recognition. J. Exp. Med. 196:1403). To determine whether SCTs of HLA-E exhibit similar peptide dependent function, an HLA-E SCT harboring the hsp60-derived peptide QMRPVSRVL (E-hsp60 SCT) was constructed. Cell-surface expression of E-hsp60 SCT was readily detectable using the conformation dependent, HLA-E-specific mAb MEM-E/6 (FIG. 6A) although the levels of E-hsp60 SCT were about 4-5 fold less than that of the HLA-E SCT containing the Cw* derived peptide VMAPRTLIL (E-Cw*03 SCT) (SEQ Id. No. 17). While E-Cw*03 SCT afforded significant protection against lysis by the human NK cell line, NKL, the effect of E-hsp60 SCT was negligible (FIG. 6B). It is unlikely that the moderate decrease in cell-surface expression of E-hsp60 SCT relative to E-Cw*03 SCT contributes to their differential efficacies in NK cell protection since a clonal line of E-Cw*03 SCT-transfected LLC-PKI cells with cell-surface expression even lower than that of E-hsp60 SCT transfectants is still resistant to lysis by NKL cells (data not shown). These experiments demonstrate that HLA-E SCTs recapitulate the peptide-dependent function of native HLA-E.

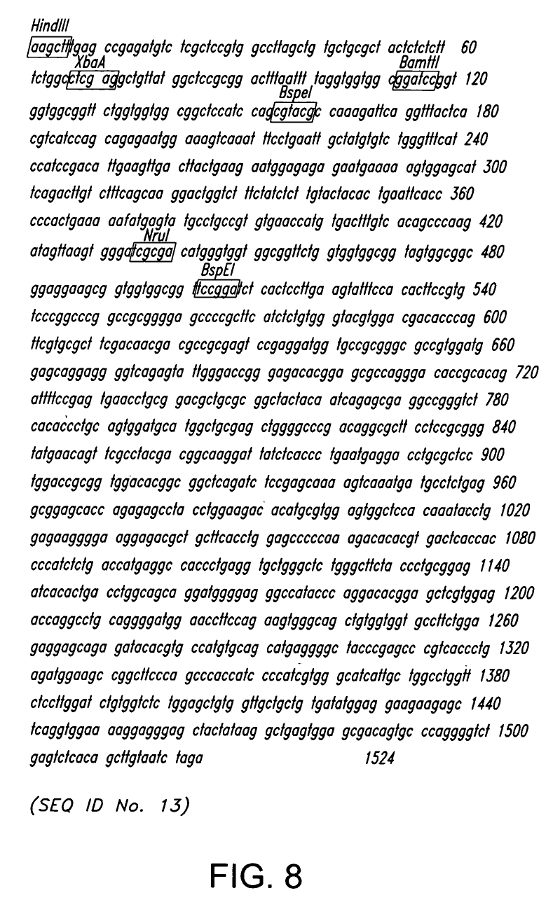

Now referring to FIG. 8, several unique restriction endonuclease recognition sites were engineered. The HindIII and XbaI sites at the 5′ and 3′ ends, respectively, facilitate cloning into a wide variety of expression vectors. The unique XhoI and BamHI sites flanking the nonamer coding sequence enable one to easily engineer a single chain trimer consisting of other nonamer sequences some of which may impart new properties to the single chain trimer. Finally, the BspEI site immediately upstream of the HLA-E coding sequence is useful for replacing the HLA-E heavy chain with other HLA class I heavy chains. Overall, the DNA encoding the HLA-E single chain trimer polypeptide provides a convenient platform to facilitate construction of other HLA class I single chain trimer-encoding genes.

The single chain HLA-E trimer polypeptide can be expressed on the surface of a pig cells. HLA class I proteins require heavy chain association with β2m and peptide nonamer for stable cell-surface expression. To determine whether HLA-E single chain trimer polypeptide would form a stable cell-surface complex, the HLA-E single chain trimer gene was transfected into a pig kidney cell line, LLC-PK1 cells, and cell-surface expression was monitored by FACs analysis using monoclonal antibodies (mAbs) specific for human β2m (mAb BM-63) and for a framework antigen of HLA class I heavy chains (mAb PA2.6). Importantly, both monoclonal antibodies recognize conformational dependent epitopes. FIG. 10 shows that HLA-E single chain trimer polypeptide can be detected on pig cell surfaces using both monoclonal antibodies. Thus, HLA-E single chain trimer polypeptide appears to fold correctly and exhibit significant stability.

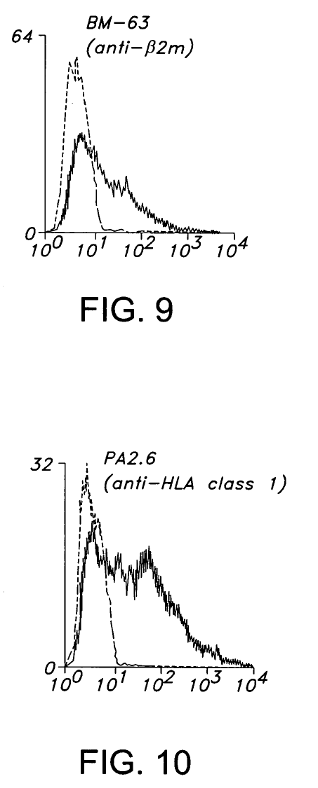

Now referring to FIGS. 9-11, FIGS. 9 and 10 are a FACs analysis of HLA-E single chain trimer polypeptide expression on LLC-PK1 cell surface. Dashed curves indicate staining of untransfected LLC-PK1 cells. Solid curves indicate staining of HLA-E single chain trimer gene-transfected LLC-PK1 cells.

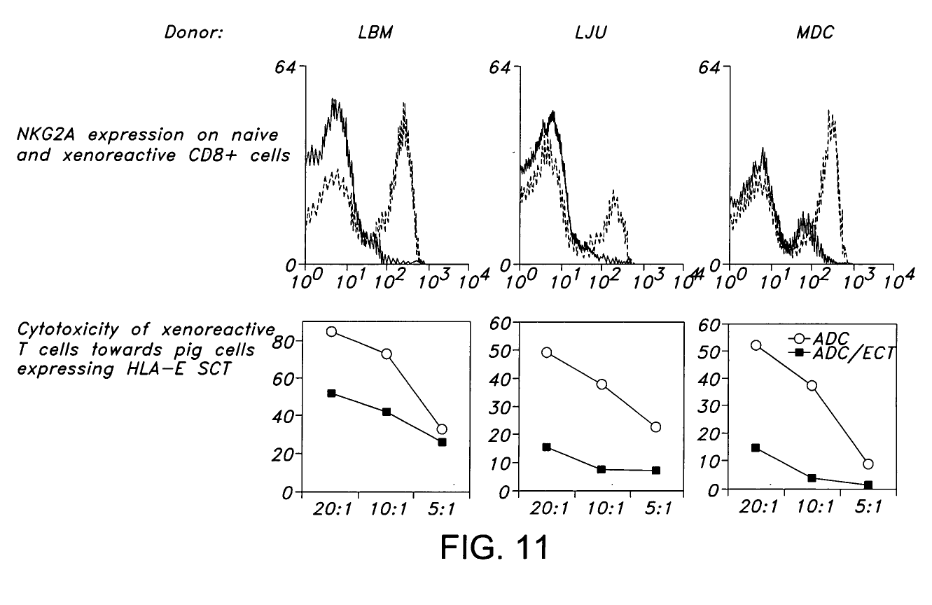

Now referring to FIG. 11, CD94/NKG2A induction on CB8+ T cells following xenoreactive stimulation and inhibition of xenoreactive CB8+ T cell-mediated lysis by HLA-E SCT-expressing AOC cells is shown. The receptor for HLA-E is CD94/NKG2A, once thought to exist only on NK cells, it is now known to be expressed on CD8+ T cells (cytotoxic T lymphocytes, CTLs) following antigenic stimulation with viruses or bacteria. It is shown in FIG. 11 that CD94/NKG2A is induced on human CTLs following xenogeneic stimulation (i.e. after co-incubation with pig aortic endothelial cells, PAECs) and moreover that PAECs expressing the HLA-E single chain trimer polypeptide are significantly less susceptible to xenoreactive CD8+ CD94/NKG2A+ T cells.

The gene encoding a human single chain trimer HLA-E polypeptide (SEQ ID No. 13) can be expressed through a variety of well known cloning procedures. Sambrook & Russell, Molecular Cloning; A Laboratory Manual, Chapter 15 & 16. (2001, 3^{rd} Ed.) (hereby specifically incorporated by reference). A method of producing a transgene encoding human single chain trimer HLA-E polypeptide in swine can be accomplished according to the methods set out in U.S. Pat. No. 6,558,663, particularly, p. 17, col. 28 showing microinjection of swine oocytes (hereby specifically incorporated by reference). The swine biological material including the transgene encoding HLA-E single chain trimer polypeptide can be introduced into a human recipient. The term swine biological material includes biological material such as cells, organs or tissues. These methods can be used to induce at least partial immunological tolerance in a recipient human to a graft obtained from a donor swine. The term “partial immunological tolerance” to a xenograft, or in other situations, means that the dose of immunosuppressive agents to be administered is comparable to dosages required for allogeneic transplants.

The xenotransplantation setting will not only provide protection against human NK cells but also CTLs as well. The HLA-E single chain trimer polypeptide can also be used in an allotransplant setting as well where CTLs are primary effectors of graft rejection. Although solid organ transplants may not benefit because of difficulties in engineering the organ, cellular allotransplants, such as bone marrow, may benefit from HLA-E single chain trimer polypeptide.

There are a couple of methods to employ the HLA-E SCT to induce tolerance to CTLs in an allogeneic setting. First, if human stem cells are used, they can be transfected with the gene encoding HLA-E SCT. These cells could then be transplanted into the human (allogeneic) recipient. Second, the HLA-E SCT gene could be engineered to encode a glyco phosphotidyl inositol (GPI) linkage signal and such a gene could be expressed in Drosophilia (fruit fly) cells. Then recombinant HLA-E SCT with GPI linkage (rHLA-E SCT-GPI) would then be purified. A recombinant single chain trimer HLA-E polypeptide can be delivered to donor cells via a glycosylphosphotidyl inositol (GPI) linkage (J. Huang, Alloantigenic Recognition of Artificial Glycosyl Phosphatidylinositol-Anchored HLA-A2.1, Molecular Immunology, Vol. 13, pp. 1017-1028, 1994, pp. 1018-1021 for methods to use glycosyl phosphatidy inositol) (hereby specifically incorporated by reference) in there use of GPI-linked HLA-A2). As described in Huang, GPI-linked HLA class I molecules can be used to “paint” the surface of cells. Therefore, rHLA-E SCT-GPI would be incubated with allogeneic human cells, tissues or whole organs prior to transplantation.

The process of introducing the transfected cells to a human recipient can be accomplished by a variety of means. Examples of suitable pharmaceutical carriers are well known in the art and include phosphate buffered saline solutions, water, emulsions, such as oil/water emulsions, various type of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. The dosage regimen will be determined by the attending physician and clinical factors. As it well known in the medical arts, dosages for any one patient depends upon many factors, including the patient's size, body surface area, age, the particular compound to be administered, sex, time and route of administration, general health, and other drugs being administered concurrently. The compositions of the invention may be administered locally or systemically. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition.

### EXAMPLES

### Example 1

### Cell Lines and Monoclonal Antibodies (mAbs)

The pig kidney epithelial cell line, LLC-PK1, and the human NK cell line, NK-92 were obtained from American Type Culture Collection (ATCC, Manassas, Va., USA). The human NK cell line, NKL, was a gift from Dr. Michael J. Robertson (Indiana University Medical Center). LLC-PK1 and NK-92 cells were maintained in and maintained in RPMI 1640 supplemented with 10% fetal calf serum, 100 μg/ml penicillin G, and 100 ug/ml streptomycin sulfate (RPMI/10%). NKL cells were propagated in the same except with 15% fetal calf serum and with 200 U/ml IL-2. IL-2 was obtained through the AIDS Research and Reference Reagent Program, Division of AIDS, NIAID, NIH from Dr. Maurice Gately, Hoffman—La Roche Inc.

The mAb PT85A which recognizes a monomorphic determinant of porcine MHC class I proteins (Davis et al., The development and analysis of species specific and cross reactive monoclonal antibodies to leukocyte differentiation antigens and antigens of the major histocompatibility complex for use in the study of the immune system in cattle and other species; Vet Immunol Immunopathol; 15: 337) was purchased from VMRD, Inc. (Pullman, Wash. USA). mAb BM-63 which is specific for human β2m was purchased from Sigma (St. Louis, Mo., USA). The HLA-E-specific mAb, 3D12, was kindly provided by Dr. Daniel Geraghty (Fred Hutchinson Cancer Research Center, Seattle Wash., USA). The HLA-E-specific mABs MEm-E/6 and MEM-E/8 (30 Menier C., B. Saez, V. Horejsi, S. Martinozzi, I. Krawice-Radanne, S. Bruel, C. Le Danff, M. Roboul, I. Hilgert, M. Rabreau, M. L. Larrad, M. Pla., E. D. Carosella, N. Rouas-Freiss 2003. Characterization of monoclonal antibodies recognizing HLA-G or HLA-E: new tools to analyze the expression of nonclassical HLA class I molecules. Hum. Immunol. 64:315) were a kind gift of Dr. Vaclay Horejsi (Institute of Molecular Genetics, Academy of Sciences of the Czech Republic, Cidenska, Czech Republic). The pan-HLA class I mAbs w6/32 and PA2.6 were obtained from ascites. The HLA-B27-specific mAb, clone HLA.ABC.m3, was purchased from Chemicon International (Temecula, Calif.). In some flow cytometric analyses, mAb UPC10 was used as isotype control (IgG₂ₐ, kappa) and PE-conjugated goat anti-mouse IgG was employed as a secondary antibody; both were purchased from Sigma (St. Louis, Mo., USA).

### Example 2

### Construction of HLA-E SCT Gene

Standard PCR and molecular cloning procedures were used to construct HLA-E single chain trimers and dimmers. Sambrook & Russell, Molecular Cloning: A Laboratory Manual, Chapter 15 & 16. (2001, 3^{rd} Ed.) (hereby specifically incorporated by reference).

To construct a gene encoding an HLA-E single chain trimer (SCT), DNA fragments encoding the β2m leader peptide linked to the VMAPRTLIL (SEQ ID NO 17) peptide, mature β2m, connecting peptide 1, and connecting peptide 2 were individually cloned into plasmids. These fragments were sequentially ligated together and subsequently fused to sequences encoding the mature HLA-E heavy chain. Oligonucleotides used in the construction of HLA-E SCT are given in the Sequence Listing.

The plasmid pB2MLP-pep contains a fragment encoding the β2m leader peptide linked to the VMAPRTLIL (SEQ ID NO 17) peptide. pB2MLP-pep was constructed by PCR amplification using the primers designated B2MF and B2MR with cloned full length human β2m cDNA as template. The PCR product was digested with BamHI and HindIII and ligated into BamHI- and HindIII-cleaved pBluescript-SK+ (Stratagene, La Jolla, Calif., USA).

pMB which contains a DNA fragment encoding mature β2m was derived from PCR amplification using primers B2MF2 and B2MR2 with cloned full length human β2m cDNA as template. The PCR product was ligated directly into pCR2.1 (Invitrogen).
pC1 contains a fragment encoding connecting peptide 1 and was derived by annealing oligonucleotides C1F and C1R and ligating the resulting double stranded DNA into EcoRV-cleaved pBluescript-SK+. pC2 contains a fragment encoding connecting peptide 2 and was made by annealing oligonucleotides C2F1, C2F2, C2R1, and C2R1, cutting the resulting double stranded DNA with HindIII and SacI followed by ligation into HindIII- and SacI-cleaved pBluescript-SK+.

The insert of pC1 was cloned into pMB2M using BsiWI and XhoI to generate pC1-MB. The insert of pC1-MB was cloned into pC2 using HindIII and NruI to create pC1-MB-C2. The insert of pC1-MB-C2 was cloned into pB2 mLP-pep using BamHI and SacI to create pLPpep-C1-MB-C2.

The final steps in the construction of the HLA-E SCT gene began with PCR amplification of mature HLA-E heavy chain-encoding sequences using HLAEF and HLAER primers with cloned full length HLA-E cDNA as template. The PCR product was digested with BspEI and XbaI and ligated with the insert of pLPpep-C1-MB-C2, excised using HindIII and BspEI, into HindIII- and XbaI-cleaved pcDNA3.1 (ClonTech, Pal Alto, Calif., USA). The HLA-E SCT gene is thus downstream of the CMV promoter and contains at its 3′ end an SV40-derived polyadenylation signal.

### Example 3

### Construction of HLA-E SCD Gene

A gene encoding an HLA-E single chain dimer (SCD), i.e. encoding the HLA-E heavy chain linked to β2m, including its leader peptide, was constructed by PCR amplification of the cloned human β2m gene using B2MF and B2MR2 primers. The resulting PCR product was digested with HindIII and EcoRI (which cleaves within the mature β2m coding sequence) and ligated in place of the HindIII, EcoRI fragment of HLA-E SCT.

### Example 4

### Transfection of LLC-PK1 Cells

LLC-PK1 cells were transiently and stably transfected. For transient transfections, 5×10⁵ cells were plated in six 10 mm plates and allowed to adhere overnight at 37 C in RPMI/10%. To identify transienty transfected cells, plasmids were co-transfected with 2 ug pEGFP-C1 (Clontech) with the total DNA for each transfection kept at 6 μg. Plasmids were resuspended in 50 ul HEPES-buffered saline (HBS) and mixed with 100 u [symbol] l HBS containing 35 ug DOTAP liposomal transfection reagent (Roche Diagnostic GmbH, Mannheim, Germany). After 20 minutes at room temperature, DNA/DOTAP complexes were added directly to the cells (in 1 ml PRM1/10%). Cells were harvested 48 hours after transfection for flow cytometric analyses as described below.

LLC-PK1 cells were stably transfected by electroporation. In brief, 2×10⁶ LLC-PK1 cells were resuspended in 200 ul RPMI/10% to which was added 20 ug DNA in 200 ul RPMI/10%. Electroporation was performed at 250 V, 960 uF and cells were replated in 5 ml RPMI/10%. Two days later, the eukaryotic antibiotic, G418 (also known as geneticin) was added to a final concentration of 1 mg/ml.

### Example 5

### Flow Cytometry

LLC-PK1 transfectants, removed from plates by trypsinization, were washed once with wash buffer (phosphate buffered saline, PBS, with 2% fetal calf serum and 0.1% NaN₃) and incubated on ice for 30-60 minutes with saturating concentrations of primary antibody. The cells were subsequently incubated with PE-conjugated goat anti-mouse IgG for 30-60 minutes on ice in wash buffer. Prior to flow cytometry cells were fixed in PBS containing 2% paraformaldehyde. Flow cytometric analyses were performed using the FACSCalibur instrument (Becton Dickinson, Franklin Lakes, N.J. USA).

### Example 6

### Cytotoxicity Assays

NK cell cytotoxicity was measured by standard ⁵¹Cr release assays with either NK-92 or NKL cells as effectors. Confluent monolayers of target cells, LLC-PK1 cells or LLC-PK1 HLA-E SCT transfectants, were incubated in RPMI/10% with 10 uCi/ml ⁵¹Cr for 16 hours at 37° C. The monolayers were washed three times with PBS prior to trypsinization. Cytotoxicity assays were performed in triplicate in 96 well U-bottom dishes using 104 target cells/well at an effector:target ratios ranging from 20:1 to 2.5:1 in a final volume of 200 ul. After various times of incubation at 37° C. (2, 4, or 6 hours), 25 ul of supernatant was removed and the radioactivity counted using a Packard gamma counter. Percent specific lysis was calculated using the formula:
[(cpmₑₓₚₑᵣᵢₘₑₙₜₐₗ−cpmₛₚₒₙₜₐₙₑₒᵤₛ/cpmₘₐₓᵢₘᵤₘ−cpmₛₚₒₙₜₐₙₑₒᵤₛ)]×100

### Example 7

### Cytokine Measurements

Equal numbers (10⁴ each) of NKL cells and untransfected or HLA-E SCT-transfected LLC-PK1 cells were co-cultured in 200 ul RPMI/10% with 100 U/ml IL-2 for 48 hours at which time 100 ul supernatant was removed and assayed for IFN-γ using an ELISA kit according to the protocol recommended by the supplier (HyCult Biotechnology, Uden, Netherlands).

### Example 8

### Generation of Xenoreactive T Lymphocytes and Analysis of Their Ability to be Inhibited by HLA-E SCT

AOC cells (Carrillo et al. 2002) were seeded in T25 flasks (1×10⁶/flask) and \tilde{y}irradiated (20,000) rads). Peripheral blood lymphocytes (PBLs) from three donors were cultured by themselves or co-cultured with the AOC cells at concentrations ranging from 1-2×10⁶/ml in 5 ml medium containing 20U/ml IL-2. Four days later, a portion (˜30%) of the PBLs were analyzed by FACS using anti-NKG2A-PE and anti-CD8-FITC mAbs (R+D Systems).

The remainder of the PBLs co-cultured with AOC cells were used as effectors in cytotoxicity assays. Targets, AOC cells or AOC cells stably transfecting HLA-E single chain trimer (“AOC/ESCT”), were labeled with ⁵¹Cr and seeded in 96 well dishes (round bottom) at 10⁴ cells/well. PBLs were added at the indicated effector:target ratios. Supernatants were counted after 4 hours co-incubation. Carrillo A., Chamorro S., Rodriguez-Gago M., Alvarez B., Molina M. J., Rodriguez-Barbosa J. I., Sanchez A., Ramirez P., Munoz A., Dominguez J., Parrilla P., Yelamos J. 2002. Isolation and characterization of immortalized porcine aortic endothelial cell lines. Vet Immunol Immunopathol. 89:91-98.

### SEQUENCE LISTING

The nucleic and amino acid sequences listed in the accompanying sequence listing are shown using standard letter abbreviations for nucleotide bases. Only one strand of each nucleic acid sequence is shown, but the complementary strand is understood as included by any reference to the displayed strand.

SEQ ID NO. 1 shows the oligonucleotide C2F1 used in the construction of HLA-E single chain trimer.

SEQ ID NO. 2 shows the oligonucleotide C2F2 used in the construction of HLA-E single chain trimer.

SEQ ID NO. 3 shows the oligonucleotide C2R2 used in the construction of HLA-E single chain trimer.

SEQ ID NO. 4 shows the oligonucleotide B2MF used in the construction of HLA-E single chain dimer and trimer.

SEQ ID NO. 5 shows the oligonucleotide B2MF2 used in the construction of HLA-E single chain trimer.

SEQ ID NO. 6 shows the oligonucleotide B2MR2 used in the construction of HLA-E single chain dimer and trimer.

SEQ ID NO. 7 shows the oligonucleotide HLAEF used in the construction of HLA-E single chain trimer.

SEQ ID NO. 8 shows the oligonucleotide HLAER used in the construction of HLA-E single chain trimer.

SEQ ID NO. 9 shows the oligonucleotide C2R1 used in the construction of HLA-E single chain trimer.

SEQ ID NO. 10 shows the oligonucleotide B2MR used in the construction of HLA-E single chain trimer.

SEQ ID NO. 11 shows the oligonucleotide C1F used in the construction of HLA-E single chain trimer.

SEQ ID NO. 12 shows the oligonucleotide C1R used in the construction of HLA-E single chain trimer.

SEQ ID NO. 13 shows the nucleotide sequence of the HLA-E single chain trimer gene.

SEQ ID NO. 14 shows the nucleotide sequence of the HLA-E single chain dimer gene.

SEQ ID NO. 15 shows the amino acid sequence of the HLA-E single chain trimer protein.

SEQ ID NO. 16 shows the amino acid sequence of the HLA-E single chain dimer protein.

SEQ ID NO. 17 shows peptides bound to inhibitory HLA-E single chain trimer polypeptides.

SEQ ID NO. 18 shows peptides bound to non-inhibitory HLA-E single chain trimer polypeptides.

1. A method to prevent Natural Killer cell-mediated rejection of a xenograft comprising:
(a) providing an isolated and purified HLA-E polypeptide comprising the amino acid sequence of SEQ ID LISTING NO. 15;
(b) administering to cells or whole animals said polypeptide; and
(c) producing an inhibitory response to said natural killer cell-mediated rejection of a xenograft.

2. A method to inhibit CTL-mediated killing of cells comprising:
(a) providing an isolated and purifed HLA-E polypeptide comprising the amino acid SEQ ID LISTING NO. 15;
(b) administering to cells or whole animals said protein; and
(c) producing an inhibitor response to said CTL-mediated killing of cells.

3. A method of inducing at least partial immunological tolerance in a recipient human to a graft obtained from a donor swine, the method comprising:
(a) introducing into the recipient human, swine biological material including a transgene encoding an isolated and purified HLA-E polypeptide comprising the amino acid sequence of SEQ ID LISTING NO. 15; and
(b) implanting the swine graft into the recipient human, wherein at least one of the cells of the swine graft express HLA-E polypeptide;
(c) wherein the introduction of said swine biological material into the recipient human results in at least partial immunological tolerance to said swine graft.

4. The method of claim 3 wherein said immunological tolerance is NK mediated.

5. The method of claim 3 wherein said immunological tolerance is CTL mediated.

6. The method of claim 3 wherein said swine biological material is a hematopoietic stem cell.

7. The method of claim 3 wherein said swine biological material selected from the group consisting of organ, cells and tissue.

8. The method of claim 3 wherein said HLA-E single chain trimer polypeptide is made from an isolated polynucleotide molecule having a sequence selected from the group consists of:
(a) a nucleotide sequence shown in SEQ ID 13;
(b) a complementary strand of a nucleotide sequence shown in SEQ ID No. 13; or
(c) fragments of a nucleotide sequence shown in SEQ ID No. 13.

9. A method of inducing at least partial CTL mediated immunologic tolerance in a recipient human to human donor cell, the method comprising:
(a) transfecting a human donor cell with isolated and purified HLA-E single chain trimer polypeptide comprising the amino acid sequence of SEQ ID LISTING NO. 15 to provide transfected cells; and
(b) introducing into the recipient human said transfected cells; wherein said introducing of said transfected cells into said recipient human results in at least partial CTL mediated immunologic tolerance to said transfected cells.

10. The method of claim 9 wherein said HLA-E single chain trimer polypeptide is made from an isolated polynucleotide molecule having a sequence selected from the group consists of:
(a) a nucleotide sequence shown in SEQ ID 13;
(b) a complementary strand of a nucleotide sequence shown in SEQ ID No. 13; or
(c) fragments of a nucleotide sequence shown in SEQ ID No. 13.
